(19) United States
(12) Patent Application Publication
(10) Pub. No.: US 2001/0034471 A1
(43) Pub. Date: Oct. 25, 2001

(54) System for the delivery of health care

(76) Inventor: Denise Runde, Mountain View, CA (US)

Correspondence Address: Pillsbury Winthrop LLP, Intellectual Property Group, East Tower, Ninth Floor, 1100 New York Avenue, N.W., San Francisco, CA, 94105, US

(21) Appl. No.: 09/772,201

(22) Filed: Jan. 29, 2001

Related U.S. Application Data
(60) Provisional application No. 60/178,886, filed on Jan. 28, 2000.

(51) Int. Cl. A61B 1/00

(52) U.S. Cl. 600/121

(57) ABSTRACT

Described is a system for the delivery of health care which comprises at least one detection device to detect or analyze information from the human body and provide sensor information or data. A receiver-transmitter is adapted to read the sensor information or data received from the detection device, and is further adapted to transfer the sensor information or data to an Internet server or web site. Software programmed to handle the sensor information or data received by the Internet server or web site determines appropriate action based on the sensor information or data and pre-established algorithms or protocols.

### FIELD OF INVENTION

As technology advances, the application of bio medical sensors, genetic screening and phenotyping along with other communication technologies will enable individuals and communities to be screened, monitored, diagnosed and treated for diseases, infections, health and medical conditions without leaving home or work place. It will also enable the screening for prevention and early detection of diseases and health and medical conditions. The Internet and wireless communication will greatly expand and enhance this capability and the accuracy of the communication between individuals and communities and the health and medical care system.

### SUMMARY OF THE INVENTION

The invention comprises a system wherein the transfer of information and data will occur directly from the individual's body and or material that has come from the body. Resulting information and or data will be transmitted through a “reader” of bio medical sensor data, micro lab or computer chip. Examples include data from an analysis or detection of body fluid, blood, tissue or other information from the body and data from biomedical sensors, genetic screening or phenotyping.

The data and information will go from the detector, sensor, medical device and reader(s) of this data and information through a receiver-transmitter “cradle” which can be capable of reading multiple sources of data and sensor information. The information can then be connected and transferred to a web site or internet server.

The data will be sent to a web site or server where its source will be identified. Software will determine eligibility of data source and reject or connect the data to the appropriate field. The software program will determine the next steps based on the information/data provided to it and a pre-established algorithm. These pre-established algorithms and protocols will have been determined by providers, payers, and/or individuals.

The invention will be better understood by reference to the following drawings and detailed description.

### BRIEF DESCRIPTION OF THE DRAWINGS

FIG. 1 is a process diagram of a preferred embodiment of the method.

### DESCRIPTION OF PREFERRED EMBODIMENTS

In a preferred embodiment the present invention comprises a detection device to detect or analyze information about the health of a human directly from the human body, from the result of information or data generated by bio medical sensors or from the analysis of micro laboratories and computer chips and provide resultant sensor information and data, a receiver-transmitter cradle and software programmed to handle the information gathered by the device.

The detection device may comprise any instrument which measures or analyzes information from the human body. The detection device can analyze information from body fluid, blood or other tissue or from information gathered from external or internal signals. For example the device could detect heat, cold, tension, muscle spasm, swelling, puffiness, presence of fluid or foreign objects, pulse, fever, pressure, range of motion, tumors, lumps, masses, blood pressure, respiratory capacity, or any other piece of information relevant to the health of a human. Instruments which might exemplify this include thermometers, glucose monitors among others.

The receiver-transmitter is a cradle that is adapted to read the sensor information resulting from the detection device. It can be adapted to receive information from more than one detection device. The receiver-transmitter is also adapted to transmit information to an internet server or web site. Specifically the receiver-transmitter transmits information from the detection device to a web site configured to accept medical information.

Software is programmed to accept the information provided to the web site or internet site by the receiver transmitter and to determine appropriate action based on a set of pre-established algorithms. The software could determine the eligibility of the source of its data and then connect eligible data to an appropriate field. The appropriate action will be determined. Algorithms and protocols which have been pre-determined can then be automatically followed or feedback can be given to the initial user.

The algorithm for determination of appropriate action is pre-established by health care providers, health care payers, such as insurance companies and the like, and the individual. The patient's medical history and relationship with the caregiver, among other things, will be taken into account. The algorithms and protocols will automatically be followed unless the software detects a problem. Appropriate action will be determined and the software will automatically take the next steps.

In a further embodiment an image may be transmitted to the web site or a care provider. The image could be transmitted to the web page or care provider along with the information and data from the detector, sensor and/or medical device via the receiver-transmitter “cradle.”

The appropriate action step will be directed by the software from which provide an automatic feedback loop of services, data, and or follow up will occur. This follow up loop will go to those indicated by the algorithms but in most instances would include the individual(s) who originally provided the data to the system and the subscriber and user of services for each transaction as appropriate. Appropriate action steps include but are not limited to contacting health professionals, sending tissue or fluid for further testing, requiring a user to avoid certain activities or the like.

If the algorithm suggests additional linkages to providers and or suppliers of health or medical care the appropriate action will be directed by the software.

In a preferred embodiment the software and service can be encrypted for security and restricted access.

In yet another embodiment the system can be used to allow small local clinics and doctor's offices to access more comprehensive assistance.

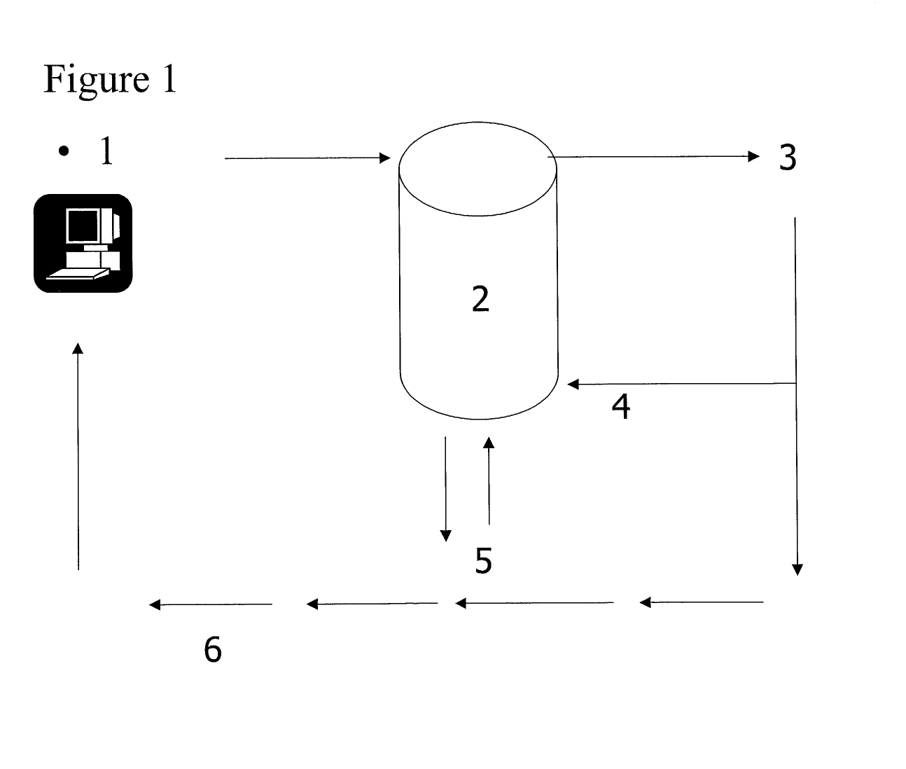

FIG. 1 shows a preferred embodiment of the process wherein a user provides information via the receiver-transmitter cradle, 1, to a website, 2. The website runs the predetermined algorithm and orders the appropriate care at step 3. In step 4 this information is returned to the website, 2, and optionally to a physician, 5. The physician provides for further appropriate orders in step 6 and also provides the information back to the website. The appropriate orders are provided to the user via the receiver-transmitter cradle, 1, or by other means.

Although the description above contains the details of preferred embodiments, these should not be construed as limiting the scope of the invention, but as merely illustrative of the invention. Indeed, variations of the invention will be readily apparent to those skilled in the art and also fall within the scope of the invention. Thus the appended claims and their legal equivalents should determine the scope of the invention.

What is claimed is:

1. A system for the delivery of health care, the system comprising:
at least one device to detect or analyze information from the human body and provide sensor information or data;
a receiver-transmitter adapted to read the sensor information or data received from the at least one device, and further adapted to transfer the sensor information or data to an Internet server or web site; and,
software programmed to handle the sensor information or data received by the Internet server or web site and determine appropriate action based on the sensor information or data and pre-established algorithms or protocols.

2. The system described in claim 1, wherein the appropriate action includes providing an automatic feedback loop of services, data, and or follow up to persons indicated by the algorithms.

3. The system described in claim 2, wherein the persons indicated by the algorithms include an individual who originally provided the data and a subscriber and user of the system.

4. The system described in claim 1, wherein the at least one device is at least one of a biomedical sensor, a microlaboratory or a computer chip.

5. The system described in claim 1, wherein the receiver-transmitter is capable of reading multiple sources of data and sensor information.

6. The system described in claim 1, wherein the Internet server or web site identifies a source of the sensor information or data and the software is programmed to determine eligibility of the source and reject or connect the sensor information or data to an appropriate field.

7. The system described in claim 1, wherein the pre-established algorithms or protocols are determined by providers, payers, and/or individuals.

8. The system described in claim 1, wherein the Internet server or web site and the software are encrypted.

9. The system described in claim 1, further comprising means for producing an image and transmitting it to the web site or Internet server.

10. A system for the delivery of health care, the system comprising:
at least one of a biomedical sensor, a microlaboratory or a computer chip to detect or analyze information from the human body;
a reader adapted to read bio medical sensor data and information from a microlaboratory or a computer chip and provide sensor information or data;
a receiver-transmitter adapted to read the sensor information or data received from the reader, and further adapted to transfer the sensor information or data to an Internet server or web site; and,
software programmed to handle the sensor information or data on the Internet server or web site and determine appropriate action based on the sensor information or data and pre-established algorithms or protocols.
